(19) United States
(12) Patent Application Publication
(10) Pub. No.: US 2004/0171035 A1
(43) Pub. Date: Sep. 2, 2004

(54) Methods and compositions for modulating P53 transcription factor

(75) Inventors: Qihong Huang, San Diego, CA (US); Sumit Chanda, La Jolla, CA (US); John B. Hogenesch, Encinitas, CA (US); Jeremy S. Caldwell, Cardiff, CA (US)

Correspondence Address: GENOMICS INSTITUTE OF THE, NOVARTIS RESEARCH FOUNDATION, 10675 JOHN JAY HOPKINS DRIVE, SUITE E225, SAN DIEGO, CA, 92121-1127, US

(73) Assignees: IRM LLC, Hamilton HM LX (BM); The Scripps Research Institute, La Jolla, CA

(21) Appl. No.: 10/704,190

(22) Filed: Nov. 7, 2003

Related U.S. Application Data
(60) Provisional application No. 60/425,351, filed on Nov. 8, 2002.

(51) Int. Cl. C12Q 1/68; C12Q 1/26

(52) U.S. Cl. 435/6; 435/25

(57) ABSTRACT

This invention provides novel p53 modulatory polypeptides. The invention also provides methods for screening modulators of p53. The methods comprise first screening test agents for modulators of a p53-modulatory polypeptide and then further screening the identified modulating agents for modulators of p53. The invention further provides methods and pharmaceutical compositions for modulating p53 bioactivities in a cell and for treating diseases and conditions mediated by abnormal cellular proliferation.

### CROSS-REFERENCE TO RELATED APPLICATIONS

This application claims the benefit of priority to U.S. Provisional Patent Application Serial No. 60/425,351 (filed Nov. 8, 2002). The full disclosure of the earlier filed application is incorporated herein by reference in its entirety and for all purposes.

### FIELD OF THE INVENTION

The present invention generally relates to methods for identifying modulators of p53 transcription factor and therapeutic applications of such modulators. More particularly, the invention pertains to novel p53 modulators that regulate bioactivities and cellular level of p53, and to methods of using such modulators to modulate p53 transcription regulating activities or cellular level in a subject.

### BACKGROUND OF THE INVENTION

The p53 transcription factor (or “p53”) is of key importance for the protection of an organism against carcinogenesis. P53 performs this function by the regulation of several cellular processes, the most important of which are apoptosis and cell-cycle progression. The p53 transcription factor is a nuclear phosphoprotein involved in the control of cell proliferation, and mutations in the p53 gene are commonly found to be associated with diverse type of human cancer (Levine et al., Nature 351: 453, 1991). Elevated p53 protein levels were observed in some human tumor lines. P53 plays a crucial role in the regulation of DNA replication at the G1/S checkpoint. Wild-type p53 allows cells to arrest in G1 so as to provide an opportunity for DNA repair prior to commencement of replicative DNA synthesis. P53 acts to reduce the incidence of cancers by mediating apoptosis in cells with activated oncogenes.

P53 exhibits DNA-binding activity (Kern et al., Science 252: 1708, 1991) and transcriptional activation properties (Fields et al., Science 249: 1046, 1990; Raycroft et al., Science 249: 1049, 1990; Bargonetti et al., Cell 65: 1083, 1991; and Agoffet al., Science 259: 84, 1993). Tetramer formation is critical to p53 ability to activate transcription (Sakaguchi et al., Biochemistry 36: 10117-24, 1997). Point mutated forms of p53 found associated with transformed cells have been observed to have lost the sequence-specific DNA binding function (Kern et al., supra; Bargonetti et al., supra; and El-Deiry et al., Nature Genetics 1: 45, 1992). Moreover, many of the mutant p53 proteins can act as dominant negatives to inhibit this activity of wild-type p53. Some viral-encoded oncoproteins (e.g., SV40 large T antigen) inhibit the DNA-binding activity of p53 apparently as a consequence of forming complexes with the p53 protein (Bargonetti et al., supra).

Modulation of p53 bioactivities (e.g., transcription regulating function) or its cellular level would affect various cellular processes and provide therapeutic means for treating a number of diseases and conditions. There is a need in the art for novel methods and compositions for modulating p53 activities and thereby inhibiting cell proliferation in tumor formation and tumor growth. The instant invention fulfills this and other needs.

### SUMMARY OF THE INVENTION

The present invention relates to novel p53-modulatory polypeptides, methods for screening modulators of the p53 transcription factor, and methods for modulating p53 activities in a cell. In one aspect, the invention provides methods for identifying an agent that modulates a p53 bioactivity. The methods comprise (a) assaying a biological activity of a p53-modulatory polypeptide, or a fragment of the polypeptide, in the presence of a test agent to identify one or more modulating agents that modulate the biological activity of the polypeptide; and (b) testing one or more of the modulating agents for ability to modulate a p53 bioactivity. In some of the methods, the testing comprises testing the modulating agents for ability to modulate p53 in regulating expression of a p53 responsive gene. In some methods, the testing comprises testing the modulating agents for ability to modulate cellular level of p53.

In some of the methods, the p53-modulatory polypeptide is a transcription factor. In some of these methods, the transcription factor is selected from the group consisting of HEY1, OSR1, HES1, AP-4, NR2F2, SFRS10, SMT3 and FLJ11339. In some other methods, the p53-modulatory polypeptide inhibits p53 activity and is selected from the group consisting of M17S2 and cathepsin B.

In some of the methods, the p53-modulatory polypeptide is a kinase and the biological activity is phosphorylation of a second polypeptide. In some other methods, the p53-modulatory polypeptide is a protease and the biological activity is proteolysis of a second polypeptide. The second polypeptide employed in such methods can be p53 or a fragment of p53.

In some of the methods, the test agent modulates cellular level of the p53-modulatory polypeptide. In some methods, the assaying of the biological activity of the p53-modulatory polypeptide occurs in a cell. The p53-modulatory polypeptide used in these methods can be expressed from a polynucleotide that has been introduced into the cell.

In some of the methods, the p53 bioactivity to be modulated is inducing expression of a second polynucleotide that is operably linked to a p53 response element. In some of these methods, the second polynucleotide encodes a reporter polypeptide. In some of the methods, the testing for ability to modulate a p53 bioactivity comprises (a) providing a cell or cell lysate that comprises the second polynucleotide that is operably linked to the p53 response element; (b) contacting the cell or cell lysate with the test agent; and (c) detecting an increase or decrease in expression of the second polynucleotide in the presence of the test agent compared to expression of the second polynucleotide in the absence of the test agent.

In some methods, the testing for ability to modulate a p53 bioactivity comprises contacting a cell or cell lysate with the test agent and determining cellular level of p53 or a fragment of p53. In some methods, the testing for ability to modulate the p53 bioactivity comprises contacting a cell or cell lysate with the test agent and determining ability of p53 to bind to a second polynucleotide that comprises a p53 response element in the cell or cell lysate.

In one aspect, the invention provides methods for identifying an agent that modulates cellular level of p53. The methods comprise (a) assaying a biological activity of a p53-modulatory polypeptide, or a fragment of the polypeptide, in the presence of a test agent to identify a modulating agent that modulates the biological activity of the polypeptide; and (b) testing the modulating agent for ability to modulate cellular level of p53. In some of these methods, the p53-modulatory polypeptide is a transcription regulatory protein and the biological activity is transcription of a second polynucleotide. The second polynucleotide can encode p53 or a fragment of p53. In some methods, the p53-modulatory polypeptide is a down-regulator of p53 and the biological activity is down-regulation of transcription of a second polynucleotide. In some methods, the p53-modulatory polypeptide is an up-regulator of p53 and the biological activity is up-regulation of transcription of a second polynucleotide.

In some of the methods, the testing comprises (a) contacting the modulating agent with a second polynucleotide operably linked to a transcription regulatory element of p53; and (b) detecting a change in cellular level of a polypeptide encoded by the second polynucleotide relative to cellular level of the polypeptide encoded by the second polynucleotide in the absence of the modulating agent. In some of these methods, the second polynucleotide encodes a reporter polypeptide. In some methods, the second polynucleotide encodes p53 or a fragment of p53.

In another aspect, the invention provides methods for identifying an agent that modulates expression of a p53 responsive gene. The methods comprise (a) contacting a test agent with a p53-modulatory polypeptide; (b) detecting a change in an activity of the p53-modulatory polypeptide relative to the activity in the absence of the test agent; and (c) detecting a change of expression level of the p53 responsive gene in the presence of the test agent identified in (b) relative to expression level of the p53 responsive gene in the absence of the test agent; thereby identifying the test agent as a modulator of expression of the p53 responsive gene.

In another aspect, the present invention provides methods of modulating a p53 bioactivity in a cell. The methods comprise administering to the cell an effective amount of a p53 modulatory polypeptide or a fragment of the p53 modulatory polypeptide, thereby modulating the p53 bioactivity. In some of these methods, the p53 modulatory polypeptide or its fragment is expressed from an expression vector that has been introduced into the cell. In still another aspect, the invention provides methods of modulating a p53 bioactivity in a cell by administering to the cell an effective amount of a p53 modulator identified in accordance with the present invention. In some of these methods, the modulating is increasing cellular level of p53.

A further understanding of the nature and advantages of the present invention may be realized by reference to the remaining portions of the specification and claims.

### DETAILED DESCRIPTION

The present invention provides novel modulators of the p53 transcription factor and methods for identifying novel p53 modulators. The invention also provides methods for modulating p53 bioactivities in a cell and for treating diseases or conditions mediated by abnormal bioactivities or cellular level of the p53 transcription factor. The following sections provide guidance for making and using the compositions of the invention, and for carrying out the methods of the invention.

I. Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more agents (e.g., two polypeptides) or combining agents and cells (e.g., a protein and a cell). Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

A “heterologous sequence” or a “heterologous nucleic acid,” as used herein, is one that originates from a source foreign to the particular host cell, or, if from the same source, is modified from its original form. Thus, a heterologous gene in a host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology. Methods for determining sequence similarity percentages (e.g., BLASTP and BLASTN using default parameters) are described herein and are generally available.

A “host cell,” as used herein, refers to a prokaryotic or eukaryotic cell that contains heterologous DNA that has been introduced into the cell by any means, e.g., electroporation, calcium phosphate precipitation, microinjection, transformation, viral infection, and/or the like.

The terms “identical”, “sequence identical” or “sequence identity” in the context of two nucleic acid sequences or amino acid sequences refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). The CLUSTAL program is well described by Higgins and Sharp (1988) Gene 73:237-244; Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment. In one class of embodiments, the polypeptides herein are at least 70%, generally at least 75%, optionally at least 80%, 85%, 90%, 95% or 99% or more identical to a reference polypeptide, e.g., a p53-modulatory polypeptide encoded by a polynucleotide in Table 1 or 2, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 95%, 99% or more identical to a reference nucleic acid, e.g., a polynucleotide in Table 1 or 2, e.g., as measured by BLASTN (or CLUSTAL, or any other available alignment software) using default parameters.

The terms “substantially identical” nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (preferably BLAST) using standard parameters. For example, the BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “isolated” means that the material is removed from its original environment (e.g., the natural environment if it is naturally occurring). For example, a naturally-occurring nucleic acid, polypeptide, or cell present in a living animal is not isolated, but the same polynucleotide, polypeptide, or cell separated from some or all of the coexisting materials in the natural system, is isolated, even if subsequently reintroduced into the natural system. Such nucleic acids can be part of a vector and/or such nucleic acids or polypeptides could be part of a composition, and still be isolated in that such vector or composition is not part of its natural environment.

The terms “nucleic acid,” “DNA sequence” or “polynucleotide” refer to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. A “polynucleotide sequence” is a nucleic acid (which is a polymer of nucleotides (A,C,T,U,G, etc. or naturally occurring or artificial nucleotide analogues) or a character string representing a nucleic acid, depending on context. Either the given nucleic acid or the complementary nucleic acid can be determined from any specified polynucleotide sequence.

The term “modulate” with respect to p53 bioactivities refers to a change in the cellular level or other biological activities of the p53 transcription factor. Modulation of p53 bioactivities can be up-regulation (i.e., activation or stimulation) or down-regulation (i.e. inhibition or suppression). For example, modulation may cause a change in cellular level of p53, enzymatic modification (e.g., phosphorylation) of p53, binding characteristics (e.g., binding to a target transcription regulatory element), or any other biological, functional, or immunological properties of p53. The change in activity can arise from, for example, an increase or decrease in expression of the p53 gene, the stability of mRNA that encodes the p53 protein, translation efficiency, or from a change in other bioactivities of the p53 transcription factor (e.g., regulating expression of a p53-responsive gene). The mode of action of a p53 modulator can be direct, e.g., through binding to the p53 protein or to genes encoding the p53 protein. The change can also be indirect, e.g., through binding to and/or modifying (e.g., enzymatically) another molecule which otherwise modulates p53 (e.g., a kinase that specifically phosphorylates p53).

The term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

The term “operably linked” refers to a functional relationship between two or more polynucleotide (e.g., DNA) segments. Typically, it refers to the functional relationship of a transcriptional regulatory sequence to a transcribed sequence. For example, a p53 promoter or enhancer sequence, is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance. A polylinker provides a convenient location for inserting coding sequences so the genes are operably linked to the p53 promoter. Polylinkers are polynucleotide sequences that comprise a series of three or more closely spaced restriction endonuclease recognition sequences.

“Plasmids” generally are designated herein by a lower case p preceded and/or followed by capital letters and/or numbers, in accordance with standard naming conventions that are familiar to those of skill in the art. Starting plasmids disclosed herein are either commercially available, publicly available on an unrestricted basis, or can be constructed from available plasmids by routine application of well known published procedures. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

The term “polypeptide” is used interchangeably herein with the terms “polypeptides” and “protein(s)”, and refers to a polymer of amino acid residues, e.g., as typically found in proteins in nature. A “mature protein” is a protein which is full-length and which, optionally, includes glycosylation or other modifications typical for the protein in a given cell membrane.

The promoter region of a gene includes the transcription regulatory elements that typically lie 5′ to a structural gene. If a gene is to be activated, proteins known as transcription factors attach to the promoter region of the gene. This assembly resembles an “on switch” by enabling an enzyme to transcribe a second genetic segment from DNA into RNA. In most cases the resulting RINA molecule serves as a template for synthesis of a specific protein, sometimes RNA itself is the final product. The promoter region may be a normal cellular promoter or an oncopromoter.

The term “recombinant” has the usual meaning in the art, and refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. When used with reference to a cell, the term indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, that has control elements that are capable of affecting expression of a structural gene that is operably linked to the control elements in hosts compatible with such sequences. Expression cassettes include at least promoters and optionally, transcription termination signals. Typically, the recombinant expression cassette includes at least a nucleic acid to be transcribed and a promoter. Additional factors necessary or helpful in effecting expression can also be used as described herein. For example, transcription termination signals, enhancers, and other nucleic acid sequences that influence gene expression, can also be included in an expression cassette.

Transcription refers to the process involving the interaction of an RNA polymerase with a gene, which directs the expression as RNA of the structural information present in the coding sequences of the gene. The process includes, but is not limited to the following steps: (1) transcription initiation, (2) transcript elongation, (3) transcript splicing, (4) transcript capping, (5) transcript termination, (6) transcript polyadenylation, (7) nuclear export of the transcript, (8) transcript editing, and (9) stabilizing the transcript.

A transcription regulatory element or sequence include, but is not limited to, a promoter sequence (e.g., the TATA box), an enhancer element, a signal sequence, or an array of transcription factor binding sites. It controls or regulates transcription of a gene operably linked to it.

A “variant” of a molecule such as a modulator of p53 is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

A “vector” is a composition for facilitating introduction, replication and/ or expression of a selected nucleic acid in a cell. Vectors include, e.g., plasmids, cosmids, viruses, YACs, bacteria, poly-lysine, etc. A “vector nucleic acid” is a nucleic acid molecule into which heterologous nucleic acid is optionally inserted which can then be introduced into an appropriate host cell. Vectors preferably have one or more origins of replication, and one or more sites into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes.” “Expression vectors” are vectors that comprise elements that provide for or facilitate transcription of nucleic acids that are cloned into the vectors. Such elements can include, e.g., promoters and/or enhancers operably coupled to a nucleic acid of interest.

### II. Identification of Novel p53-Modulatory Polypeptides

Huma p53 gene encodes a 393 amino acid residue, 53 kD phosphoprotein. The protein is divided structurally and functionally into four domains. The first 42 amino acids at the N-terminus constitute a transcriptional activation machinery in positively regulating gene expression. Amino acid residues 13-23 in the p53 protein are identical in a number of diverse species and certain amino acids in this region have been shown to be required for transcriptional activation by the protein in vivo. The sequence-specific DNA binding domain of p53 is localized between amino acid residues 102 and 292. The native p53 is a tetramer in solution, and amino acid residues 324-355 are required for this oligomerization of the protein. The C-terminal 26 amino acids form an open domain composed of nine basic amino acid residues that bind to DNA and RNA readily with some sequence or structural preferences. There is evidence that the p53 protein requires a structural change to activate it for sequence specific binding to DNA. Deletion of the C-terminus domain activates site-specific DNA binding by the central domain.

As used in the present invention, the consensus binding sites on a target gene that is regulated by the p53 transcription factor (i.e., a p53 responsive gene) are interchangeably termed “p53 recognition sequences,” “p53 response elements,” or “p53 binding sites.” These sequences are found in many p53 responsive genes and usually have a consensus p53 DNA binding motif, e.g., TGCCT repeats (Kley et al., Nucleic Acids Res. 20: 4083-7, 1992; and Jackson et al., Gene Expr. 5: 19-33, 1995). Example of genes containing p53-binding sequences have been described in the art, e.g., in Yuan et al., Biochem Biophys Res Commun 191: 662-8, 1993; Zauberman et al., Oncogene 10: 2361-6, 1995; Jackson et al., FEBS Lett. 406: 271-4, 1997; Grand et al., Oncogene 18: 955-65, 1999; and Brazda et al., Biochem Biophys Res Commun. 267: 934-9, 2000.

The present invention provides novel protein or polypeptide modulators that modulate p53. Utilizing an expression vector which expresses a reporter gene under the control of a p53 responsive sequence, a number of polynucleotides were identified which modulate expression of the reporter gene when the expression vector and the polynucleotides were co-transfected into a host cell (see Example 1 below). Table 1 and Table 2 respectively list exemplary polynucleotides encoding p53-modulatory polypeptides that down-regulate or up-regulate the reporter gene expression. As shown in the Tables, the novel p53-modulatory polypeptides include very diversified classes of proteins, including transcription factors, kinases, aminoacylases, RNA binding proteins, receptor polypeptides, and etc.

In addition to identifying the novel p53-modulatory molecules, the present inventors also found that some of the p53 modulators, e.g., HES and HES-related protein HEY1, activate p53 through repression of hDM2 transcription (see Examples 2 and 3 below). hDM2 is a major regulator of p53, which inhibits p53 activity by targeting p53 ubiquitination, inhibiting acetylation of p53 and transporting p53 to the cytoplasm. It was shown by the present inventors that ectopic expression of these bHLH transcription factors in both zebrafish and avian developmental systems resulted in p53 overexpression. Furthermore, Ras and Myc oncogene mediated transformation of mouse embryonic fibroblasts (MEFs), which is inhibited by p53, was correspondingly abrogated by expression of HEY1 and HES. These results reveal that these transcriptional inhibitors are members of an evolutionary conserved network of p53 function.

The p53-modulatory polypeptides identified by the present inventors can operate with a number of mechanisms in modulating p53. For example, they can modulate upstream pathways leading to p53 activation (e.g., a kinase pathway). P53 is activated by and respond to very diversified signals, e.g., cellular stress, ribonucleotide triphosphate depletion, DNA damage, and hypoxia (see, e.g., Almog et al., Biochim Biophys Acta, 1378:R43-R54, 1998; and Oren et al., Biochim Biophys Acta, 1288: R13-R19, 1996). Modulation of the reporter gene expression by the p53 modulatory polypeptides shown in Tables 1 and 2 could also be due to a direct effect on the p53 responsive element in the expression construct and transcription of the reporter gene. Thus, the p53-modulatory polypeptides can be transcriptional regulators or co-regulators of p53 responsive genes.

Further, the modulation could be the result of altered activities of endogenous p53 that in turn modulates expression of the reporter gene. The p53-modulatory polypeptides of the present invention could exert regulatory function on expression of the p53 gene and cellular level of the p53 protein, e.g., as transcriptional regulators or co-regulators of the p53 gene. They can stimulate or inhibit expression of the p53 gene or otherwise alter cellular level of the p53 protein by, e.g., modulating events relating to transcription of the p53 gene, modulating post-transcriptional processing, modulating translation of p53, modulating post-translational modification, or modulating stability or proteolysis of the p53 protein. As shown in the Examples below, some of the p53-modulatory polypeptides (e.g., HEY1, OSR1, HES1, AP-4) are transcription factors that positively regulate p53 protein levels in cells.

Other than modulating cellular level of endogenous p53, the p53-modulatory polypeptides can also act by modulating other biological activities of the p53 transcription factor that are necessary for or involved in the transcription regulating function of p53. For example, they can modulate phosphorylation of the p53 protein. Phosphorylation of p53 is implicated in different bioactivities of p53 (see, e.g., Steegenga et al., J Mol Biol 263: 103-13, 1996; Milczarek et al., Life Sci. 60: 1-11, 1997; Suzuki et al., Biochem Biophys Res Commun, 183: 1175-83, 1992; Milne et al., Nucleic Acids Res. 20: 5565-70, 1992; Hall et al., Nucleic Acids Res. 24: 1119-26, 1996; and Mayr et al., Cancer Res. 55: 2410-7, 1995). A number of protein kinases are known to phosphorylate p53, e.g., casein kinase II (Milne et al., Nucleic Acids Res. 20: 5565-70, 1992), mitogen-activated protein kinases (Milne et al., J Biol Chem 269: 9253-60, 1994), protein kinases CK2 (Cell Mol Biol Res 40: 555-61, 1994), protein kinases C (Chernov et al., Proc Natl Acad Sci USA 95: 2284-9, 1998), and DNA-PK (Lees-Miller et al., Mol. Cell. Biol. 12: 5041-5049, 1992).

Phosphorylation of p53 is important for its DNA binding activity. For example, it was reported that hyperphosphorylation of p53 has different effects on its DNA-binding activity, depending on the phosphorylation sites and the binding motif (Hecker et al., Oncogene 12: 953-61, 1996). It was also shown that stabilization and activation of p53 are regulated independently by different phosphorylation events (Chemov et al., Proc Natl Acad Sci USA 95: 2284-9, 1998). For example, phosphorylation of serine 392 stabilizes p53 tetramer (Sakaguchi et al., Biochemistry 36: 10117-24, 1997). It was suggested that p53 in undamaged cells may be largely monomeric, while tetramer formation through phosphorylation of Ser392 provides a switch that activates p53 as a transcription factor in response to DNA damage. Phosphorylation of p53 can also affect the activity of the transcription-activation domain of p53 (Steegenga et al., J Mol Biol, 263: 103-13, 1996).

The p53-modulatory polypeptides can also modulate p53 interaction with other transcription factors or proteins that are involved in transcription regulation of p53 responsive genes. A number of proteins are known to bind to p53 and modulate p53 activities. For example, suppressor c-Ab1, which interacts with p53 in response to DNA damage, stimulates p53 DNA binding and tetramerization (e.g., Nie et al., Mol Cell Biol. 20: 741-8, 2000); replication protein A inhibits p53 sequence-specific DNA binding by forming a complex with p53 (Miller et al., Mol Cell Biol 17: 2194-201, 1997). The p53-modulatory polypeptides of the present invention can modulate p53 cellular activities by indirectly modulate any of these proteins or factors that interact with p53.

TABLE 1
Polynucleotides encoding down-regulating p53-modulatory polypeptides

| | GenBank Acc. | Description of the polynucleotide | | Fold of |
|---|---|---|---|---|
| | No. | sequence and encoded polypeptide | Additional reference | modulation |
| 1 | NM_000140.1 | Homo sapiens ferrochelatase | Biochem. Biophys. Res. | 5.2× |
| | | (protoporphyria) (FECH), nuclear gene | Commun. 173 (2), 748-755 | |
| | | encoding mitochondrial protein | (1990) | |
| 2 | NM_022783.1 | Homo sapiens hypothetical protein | Genome Res. 11: 422-435 | 3.7× |
| | | FLJ12428 (FLJ12428) | (2001) | |
| 3 | BC004118 | Homo sapiens, clone MGC: 11170 | | 2× |
| | | IMAGE: 3843148 | | |
| 4 | AF415191.1 | Homo sapiens calcium/calmodulin- | Diabetologia 45: 580-583 | 2.9× |
| | | dependent protein kinase II gamma | (2002) | |
| | | (CAMK2G) gene, exons 16a and 16b | | |
| 5 | NM_014371.1 | Homo sapiens neighbor of A-kinase | | 3.2× |
| | | anchoring protein 95 (NAKAP95) | | |
| 6 | BC000545 | Homo sapiens, aminoacylase 1, clone | | 3.2× |
| | | MGC: 2251 | | |
| 7 | AF281074 | Homo sapiens neuropilin 2 (NRP2) gene | Genomics 70: 211-222 | 3.6× |
| | | | (2000) | |
| 8 | XM_036507.5 | Homo sapiens E74-like factor 3 (ets | | 4.7× |
| | | domain transcription factor, epithelial- | | |
| | | specific) (ELF3) | | |
| 9 | BC000892 | Homo sapiens, HSPC154 protein, clone | | 3× |
| | | MGC: 4897 | | |
| 10 | NM_001280.1 | Homo sapiens cold inducible RNA | J. Cell Biol. 137: 899-908 | 2.9× |
| | | binding protein (CIRBP) | (1997) | |
| 11 | NM_006556.2 | Homo sapiens phosphomevalonate | J. Biol. Chem. 271: | 2.6× |
| | | kinase (PMVK) | 17330-17334 (1996) | |
| 12 | BC005948 | Homo sapiens, small muscle protein, X- | | 3.2× |
| | | linked, clone MGC: 14584 | | |
| 13 | XM_064499.1 | Homo sapiens LOC125238 | | 3.3× |
| | | (LOC125238) | | |
| 14 | XM_035662.2 | Homo sapiens cathepsin B (CTSB) | | 5.5× |
| 15 | XM_059572.5 | Homo sapiens similar to CG9886 gene | | 3× |
| | | product (LOC132158) | | |
| 16 | BC000864 | Homo sapiens, Sjogren's syndrome | | 3.5× |
| | | nuclear autoantigen 1, clone MGC: 5078 | | |
| 17 | NM_005899.2 | Homo sapiens membrane component, | DNA Res. 1: 223-229 | 5.6× |
| | | chromosome 17, surface marker 2 | (1994) | |
| | | (ovarian carcinoma antigen CA125) | | |
| | | (M17S2), transcript variant 1 | | |
| 18 | AK000694 | Homo sapiens cDNA FLJ20687 fis, | | 3.5× |
| | | clone KAIA302, highly similar to | | |
| | | AF039702 Homo sapiens antigen NY- | | |
| | | CO-43 mRNA | | |
| 19 | NM_002347.1 | Homo sapiens lymphocyte antigen 6 | Genomics 53: 365-368 | 7.2× |
| | | complex, locus H (LY6H) | (1998) | |
| 20 | BC005307.1 | Homo sapiens, kallikrein 3, (prostate | | 3.1× |
| | | specific antigen), clone MGC: 12378 | | |
| 21 | NM_022783.1 | Homo sapiens hypothetical protein | Genome Res. 11: 422-435 | 3.7× |
| | | FLJ12428 (FLJ12428) | (2001) | |
| 22 | AK002201 | Homo sapiens cDNA, weakly similar to | | |
| | | Homo sapiens myosin-IXb splice | | |
| | | variant mRNA (FLJ11339). | | |

TABLE 2
Polynucleotides encoding up-regulating p53-modulatory polypeptides

| | GenBank | Description of the polynucleotide sequence | | Fold of |
|---|---|---|---|---|
| | Acc. No. | and encoded polypeptide | Additional reference | modulation |
| 1 | NM_134323.1 | Homo sapiens TAR (HIV) RNA binding | Science 251: 1597-1600 | 22.2× |
| | | protein 2 (TARBP2), transcript variant 1 | (1991) | |
| 2 | BC003353 | Homo sapiens, Similar to CG5057 gene | | 85× |
| | | product | | |
| 3 | XM_009805.6 | Homo sapiens SMT3 suppressor of mif two | | 14.4× |
| | | 3 homolog 1 (yeast) (SMT3) | | |
| 4 | NM_012258.2 | Homo sapiens hairy/enhancer-of-split | Biochem. Biophys. | 59× |
| | | related with YRPW motif 1 (HEY1) | Res. Commun. 260: | |
| | | | 459-465 (1999) | |
| 5 | XM_059439.3 | Homo sapiens similar to odd-skipped related | | 15.7× |
| | | 1 (Drosophila); odd-skipped related gene; | | |
| | | odz (odd Oz/ten-m) homolog (Drosophila) | | |
| | | related 1 (LOC130497) (OSR1) | | |
| 6 | XM_008017.4 | Homo sapiens transcription factor AP-4 | | 34.4× |
| | | (activating enhancer binding protein 4) | | |
| | | (AP4) | | |
| 7 | NM_004593.1 | Homo sapiens splicing factor, | Genomics 33: 151-152 | 42.9× |
| | | arginine/serine-rich 10 (transformer 2 | (1996) | |
| | | homolog, Drosophila) (SFRS10) | | |
| 8 | XM_034935.2 | Homo sapiens similar to spindlin-like | | 64× |
| | | protein 2 (LOC203441) | | |
| 9 | NM_022462.1 | Homo sapiens hypothetical protein | Biochem. Biophys. | 24.3× |
| | | FLJ14033 similar to hypoxia inducible | Res. Commun. 287: | |
| | | factor 3, alpha subunit (HIF-3A) | 808-813 (2001) | |
| 10 | AK057886 | Homo sapiens cDNA FLJ25157 fis, clone | | n/d |
| | | CBR08008, highly similar to ubiquitin- | | |
| | | conjugating enzyme E2-23 KDA (EC | | |
| | | 6.3.2.19) | | |
| 11 | XM_046581.5 | Homo sapiens KIAA1511 protein | | 148× |
| | | (KIAA1511) | | |
| 12 | NM_021005.1 | Homo sapiens nuclear receptor subfamily 2, | Gene Expr. 1 (3), | 142× |
| | | group F, member 2 (NR2F2) | 207-216 (1991) | |
| 13 | XM_170886.1 | Homo sapiens HSPC189 protein (HSPC189) | | 9.8× |
| 14 | BC009203 | Homo sapiens, clone MGC: 15393 | | 71× |
| 15 | NM_005524 | Homo sapiens hairy and enhancer of split 1, | Nature 423 (6942), | 5× |
| | | (Drosophila), mRNA (HES1) | 838-842 (2003) | |
| 16 | AK002201 | Homo sapiens cDNA, weakly similar to | | |
| | | Homo sapiens myosin-IXb splice variant | | |
| | | mRNA (FLJ11339). | | |

### III. Methods for Screening Modulators of p53

The p53-modulatory polypeptides described above provide novel targets for screening modulators (agonists or antagonists) of the p53 transcription factor. The novel p53 modulators can be used to modulate transcription regulation of p53 responsive genes. The expression of a p53 responsive gene can be positively or negatively regulated to provide, respectively, for increased or decreased production of the protein whose expression is mediated by a p53 response element. Furthermore, genes that do not have p53 response elements in their wild type form can be placed under the control of p53 by inserting a p53 binding site in an appropriate position, using techniques known to those skilled in the art. Thus, expression of such genes can also be modulated by p53 modulators of the present invention.

A. General Scheme and Assay Systems

Employing the novel p53-modulatory polypeptides described above, the present invention provides methods for screening agents or compounds that modulate activities of the p53 transcription factor. Various biochemical and molecular biology techniques well known in the art can be employed to practice the present invention. Such techniques are described in, e.g., Sambrook et al., Molecular Cloning: A Laboratory Manual, Cold Spring Harbor Press, N.Y., Second (1989) and Third (2000) Editions; and Ausubel et al., Current Protocols in Molecular Biology, John Wiley & Sons, Inc., New York (1987-1999).

In some methods, test agents are first assayed for their ability to modulate a biological activity of a p53-modulatory polypeptide (“the first assay step”). Modulating agents thus identified are then subject to further screening for ability to modulate a biological activity of the p53 transcription factor, typically in the presence of the p53-modulatory polypeptide (“the second testing step”). Depending on the p53-modulatory polypeptide employed in the method, modulation of different biological activities of the p53-modulatory polypeptide can be assayed in the first step. For example, a test agent can be assayed for binding to the p53-modulatory polypeptide. The test agent can be assayed for activity to modulate expression level of the p53-modulatory polypeptide, e.g., transcription or translation. The test agent can also be assayed for activities in modulating cellular level or stability of the p53-modulatory polypeptide, e.g., post-translational modification or proteolysis. For example, modulation of p53 stability by a test agent can be examined with a p53-GFP fusion construct using methods as described in, e.g., Akgul et al., FEBS Lett. 478: 72-6, 2000.

If the p53-modulatory polypeptide has a known or well established biological or enzymatic function (e.g., kinase activity, protease activity, or DNA-binding activity), the biological activity monitored in the first screening step can be the specific biochemical or enzymatic activity of the p53-modulatory polypeptide. In an exemplary embodiment, the p53-modulatory polypeptide is a kinase (e.g., encoded by a polynucleotide with accession number AF415191.1 or NM_{—}006556.2 in Table 1), and test agents are first screened for modulating the kinase's activity in phosphorylating a substrate. The substrate can be a polypeptide known to be phosphorylated by the kinase. The substrate can also be the p53 transcription factor or a fragment harboring the kinase binding site and the phosphorylation site (e.g., a functional derivative of the p53 transcription factor).

Once test agents that modulate the p53-modulatory polypeptides are identified, they are typically further tested for ability to modulate the p53 transcription factor. The test agents can be further tested for ability to modulate expression or cellular level of p53 or fragment thereof. For example, some of the p53-modulatory molecules were shown to alter p53 protein level (e.g., HEY1, OSR1, AP-4, HES1, SMT3, FLJ11339, NR2F2, and SRSF10; see Examples 2 and 3 below). When these molecules are employed to screen test agents, modulating agents identified in the first screening step can be further tested for ability to regulate cellular level of the p53 transcription factor. Alternatively, the test agents can be further tested for activity on modulating transcription regulating function of p53, e.g., binding to a p53 recognition sequence or promoting expression of a gene under the control of a p53 binding sequence (i.e., a p53 responsive gene).

As noted above, the p53-modulatory polypeptides identified by the present inventors can modulate cellular level of p53 or transcription-regulating functions of p53. If a test agent identified in the first screening step modulates cellular level (e.g., by altering transcription activity) of the p53-modulatory polypeptide, it would indirectly modulate the p53 transcription factor. For example, if the p53-modulatory polypeptide (e.g., a kinase) modulates p53 activities by specifically phosphorylating p53, a test agent which alters cellular level of the p53-modulatory kinase would indirectly also modulate p53 activities. Similarly, if the p53-modulatory polypeptide modulates cellular level of p53 (e.g., HEY1, OSR1, AP-4, HES1, SMT3, FLJ11339, NR2F2, or SRSF10), a test agent that modulates cellular level of the p53-modulatory polypeptide would indirectly alter cellular level of p53.

On the other hand, if a test agent modulates an activity other than cellular level of the p53-modulatory polypeptide, then the further testing step is needed to confirm that their modulatory effect on the p53-modulatory polypeptide would indeed lead to modulation of p53 activities (e.g., cellular level of p53 or transcription regulating function of p53). For example, a test agent which modulates phosphorylation activity of a p53-modulatory polypeptide needs to be further tested in order to confirm that modulation of phosphorylation activity of the p53-modulatory polypeptide can result in modulation of p53 transcription regulating function or p53 cellular level.

In both the first assaying step and the second testing step, either intact p53-modulatory polypeptide and p53 or their fragments, analogs, or functional derivatives can be used. The fragments that can be employed in these assays usually retain one or more of the biological activities of the p53-modulatory polypeptide (e.g., kinase activity if the p53-modulatory employed in the first assaying step is a kinase) and p53 (e.g., binding to a p53 recognition sequence). Fusion proteins containing such fragments or analogs can also be used for the screening of test agents. Functional derivatives of p53-modulatory polypeptide and p53 usually have amino acid deletions and/or insertions and/or substitutions while maintaining one or more of the bioactivities and therefore can also be used in practicing the screening methods of the present invention.

A functional derivative of p53-modulatory polypeptide or p53 can be prepared from a naturally occurring or recombinantly expressed protein by proteolytic cleavage followed by conventional purification procedures known to those skilled in the art. Alternatively, the functional derivative can be produced by recombinant DNA technology by expressing only fragments of a p53-modulatory polypeptide or p53 that retain one or more of their bioactivities.

A variety of routinely practiced assays can be used to identify test agents that modulate a p53-modulatory polypeptide or p53. Preferably, the test agents are screened with a cell based assay system. For example, in a typical cell based assay for screening p53 modulators (i.e., the second screening step), a construct comprising a p53 transcription regulatory element operably linked to a reporter gene is introduced into a host cell system. The reporter gene activity (e.g., an enzymatic activity) in the presence of a test agent can be determined and compared to the activity of the reporter gene in the absence of the test agent. An increase or decrease in the activity identifies a modulator of p53. The reporter gene can encode any detectable polypeptide (response or reporter polypeptide) known in the art, e.g., detectable by fluorescence or phosphorescence or by virtue of its possessing an enzymatic activity. The detectable response polypeptide can be, e.g., luciferase, alpha-glucuronidase, alpha-galactosidase, chloramphenicol acetyl transferase, green fluorescent protein, enhanced green fluorescent protein, and the human secreted alkaline phosphatase.

In the cell-based assays, the test agent (e.g., a peptide or a polypeptide) can also be expressed from a different vector that is also present in the host cell. In some methods, a library of test agents are encoded by a library of such vectors (e.g., a cDNA library; see the Example below). Such libraries can be generated using methods well known in the art (see, e.g., Sambrook et al. and Ausubel et al., supra) or obtained from a variety of commercial sources.

In addition to cell based assays described above, modulators of p53 can also be screened with non-cell based methods. These methods include, e.g., mobility shift DNA-binding assays, methylation and uracil interference assays, DNase and hydroxy radical footprinting analysis, fluorescence polarization, and UV crosslinking or chemical cross-linkers. For a general overview, see, e.g., Ausubel et al., supra (chapter 12, DNA-Protein Interactions). One technique for isolating co-associating proteins, including nucleic acid and DNA/RNA binding proteins, includes use of UV crosslinking or chemical cross-linkers, including e.g., cleavable cross-linkers dithiobis (succinimidylpropionate) and 3,3′-dithiobis (sulfosuccinimidyl-propionate); see, e.g., McLaughlin (1996) Am. J. Hum. Genet. 59:561-569; Tang (1996) Biochemistry 35:8216-8225; Lingner (1996) Proc. Natl. Acad. Sci. USA 93:10712; and Chodosh (1986) Mol. Cell. Biol 6:4723-4733.

B. Test Agents

Test agents that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecules (e.g., molecules with a molecular weight of not more than about 1,000). Preferably, high throughput assays are adapted and used to screen for such small molecules. In some methods, combinatorial libraries of small molecule test agents as described above can be readily employed to screen for small molecule modulators of p53. A number of assays are available for such screening, e.g., as described in Schultz (1998) Bioorg Med Chem Lett 8:2409-2414; Weller (1997) Mol Divers. 3:61-70; Fernandes (1998) Curr Opin Chem Biol 2:597-603; and Sittampalam (1997) Curr Opin Chem Biol 1:384-91.

Libraries of test agents to be screened with the claimed methods can also be generated based on structural studies of the p53-modulatory polypeptides, their fragments or analogs. Such structural studies allow the identification of test agents that are more likely to bind to the p53-modulatory polypeptides. The three-dimensional structure of a p53-modulatory polypeptide can be studied in a number of ways, e.g., crystal structure and molecular modeling. Methods of studying protein structures using x-ray crystallography are well known in the literature. See Physical Bio-chemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of p53-modulatory polypeptides' structures provides another means for designing test agents for screening p53 modulators. Methods of molecular modeling have been described in the literature, e.g., U.S. Pat. No. 5,612,894 entitled “System and method for molecular modeling utilizing a sensitivity factor”, and U.S. Pat. No. 5,583,973 entitled “Molecular modeling method and system”. In addition, protein structures can also be determined by neutron diffraction and nuclear magnetic resonance (NMR). See, e.g., Physical Chemistry, 4th Ed. Moore, W. J. (Prentice-Hall, New Jersey 1972), and NMR of Proteins and Nucleic Acids, K. Wuthrich (Wiley-Interscience, New York 1986).

Modulators of the present invention also include antibodies that specifically bind to a p53-modulatory polypeptide in Tables 1 and 2. Such antibodies can be monoclonal or polyclonal. Such antibodies can be generated using methods well known in the art. For example, the production of non-human monoclonal antibodies, e.g., murine or rat, can be accomplished by, for example, immunizing the animal with a p53-modulatory polypeptide or its fragment (See Harlow & Lane, Antibodies, A Laboratory Manual, Cold Spring Harbor Laboratory Press, Cold Spring Harbor N.Y.). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to a p53-modulatory polypeptide of the present invention.

Human antibodies against a p53-modulatory polypeptide can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using a p53-modulatory polypeptide or its fragment.

C. Screening Test Agents that Modulate p53-Modulatory Polypeptides

A number of assay systems can be employed to screen test agents for modulators of a p53-modulatory polypeptide. As noted above, the screening can utilize an in vitro assay system or a cell-based assay system. In this screening step, test agents can be screened for binding to the p53-modulatory polypeptide, altering cellular level of the p53-modulatory polypeptide, or modulating other biological activities of the p53-modulatory polypeptide.

1. Binding of Test Agents to a p53-Modulatory Polypeptide

In some methods, binding of a test agent to a p53-modulatory polypeptide is determined in the first screening step. Binding of test agents to a p53-modulatory polypeptide can be assayed by a number of methods including e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. The test agent can be identified by detecting a direct binding to the p53-modulatory polypeptide, e.g., co-immunoprecipitation with the p53-modulatory polypeptide by an antibody directed to the p53-modulatory polypeptide. The test agent can also be identified by detecting a signal that indicates that the agent binds to the p53-modulatory polypeptide, e.g., fluorescence quenching.

Competition assays provide a suitable format for identifying test agents that specifically bind to a p53-modulatory polypeptide. In such formats, test agents are screened in competition with a compound already known to bind to the p53-modulatory polypeptide. The known binding compound can be a synthetic compound. It can also be an antibody, which specifically recognizes the p53-modulatory polypeptide, e.g., a monoclonal antibody directed against the p53-modulatory polypeptide. If the test agent inhibits binding of the compound known to bind the p53-modulatory polypeptide, then the test agent also binds the p53-modulatory polypeptide.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, “Antibodies, A Laboratory Manual,” Cold Spring Harbor Press (1988)); solid phase direct label RIA using ¹²⁵I label (see Morel et al., Mol. Immunol. 25(1):7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified polypeptide bound to a solid surface or cells bearing either of these, an unlabelled test agent and a labeled reference compound. Competitive inhibition is measured by determining the amount of label bound to the solid surface or cells in the presence of the test agent. Usually the test agent is present in excess. Modulating agents identified by competition assay include agents binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize a p53-modulatory polypeptide or its fragments onto a solid phase matrix. The solid phase matrix is then put in contact with test agents, for an interval sufficient to allow the test agents to bind. Following washing away any unbound material from the solid phase matrix, the presence of the agent bound to the solid phase allows identification of the agent. The methods can further include the step of eluting the bound agent from the solid phase matrix, thereby isolating the agent. Alternatively, other than immobilizing the p53-modulatory polypeptide, the test agents are bound to the solid matrix and the p53-modulatory polypeptide molecule is then added.

Soluble assays include some of the combinatory libraries screening methods described above. Under the soluble assay formats, neither the test agents nor the p53-modulatory polypeptide are bound to a solid support. Binding of a p53-modulatory polypeptide or fragment thereof to a test agent can be determined by, e.g., changes in fluorescence of either the p53-modulatory polypeptide or the test agents, or both. Fluorescence may be intrinsic or conferred by labeling either component with a fluorophor.

In some binding assays, either the p53-modulatory polypeptide, the test agent, or a third molecule (e.g., an antibody against the p53-modulatory polypeptide) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵I, ³²P, ³⁵S) or a chemiluminescent or fluorescent group. Similarly, the detectable group can be a substrate, cofactor, inhibitor or affinity ligand.

2. Agents Modulating other Activities of p53-Modulatory Polypeptides

Binding of a test agent to a p53-modulatory polypeptide provides an indication that the agent can be a modulator of the p53-modulatory polypeptide. It also suggests that the agent may modulate p53 bioactivities (e.g., by binding to and modulate the p53-modulatory polypeptide which in turn acts on p53). Thus, a test agent that binds to a p53-modulatory polypeptide can be further tested for ability to modulate p53 activities (i.e., in the second testing step outlined above).

Alternatively, a test agent that binds to a p53-modulatory polypeptide can be further examined to determine its activity on the p53-modulatory polypeptide. The existence, nature, and extent of such activity can be tested by an activity assay. Such an activity assay can confirm that the test agent binding to the p53-modulatory polypeptide indeed has a modulatory activity on the p53-modulatory polypeptide. More often, such activity assays can be used independently to identify test agents that modulate activities of a p53-modulatory polypeptide (i.e., without first assaying their ability to bind to the p53-modulatory polypeptide). In general, such methods involve adding a test agent to a sample containing a p53-modulatory polypeptide in the presence or absence of other molecules or reagents which are necessary to test a biological activity of the p53-modulatory polypeptide (e.g., kinase activity if the p53-modulatory polypeptide is a kinase), and determining an alteration in the biological activity of the p53-modulatory polypeptide. In addition to assays for screening agents that modulate an enzymatic or other biological activities of a p53-modulatory polypeptide, the activity assays also encompass in vitro screening and in vivo screening for alterations in expression or cellular level of the p53-modulatory polypeptide.

In an exemplary embodiment, the p53-modulatory polypeptide is a kinase, and the test agent is examined for ability to modulate the kinase activity of the p53-modulatory polypeptide. Methods for monitoring kinase activity and p53 phosphorylation are described, e.g., in Ashcroft et al., Mol Cell Biol. 19: 1751-8, 1999; Hecker et al., Oncogene 12: 953-61, 1996; Chemov et al., Proc Natl Acad Sci USA 95: 2284-9, 1998; Sakaguchi et al., Biochemistry 36: 10117-24, 1997; Steegenga et al., J Mol Biol, 263: 103-13, 1996. Any of these methods can be employed to assay modulatory effect of a test agent on a p53-modulatory polypeptide (e.g., one encoded by a polynucleotide with accession number AF415191.1 or NM_{—}006556.2 in Table 1).

D. Screening for Agents that Modulate p53

Once a modulating agent has been identified to bind to a p53-modulatory polypeptide and/or to modulate a biological activity (including cellular level) of the p53-modulatory polypeptide, it can be further tested for ability to modulate bioactivities of the p53 transcription factor. Modulation of p53 bioactivities by the modulating agent is typically tested in the presence of the p53-modulatory polypeptide. When a cell-based screening system is employed, the p53-modulatory polypeptide can be expressed from an expression vector that has been introduced into a host cell. P53 or a p53 fragment can be expressed from a second expression vector. Alternatively, the p53 transcription factor can be supplied endogenously by the host cell in the screening system.

1. p53 Bioactivities to be Monitored

Unless otherwise specified, modulation of bioactivities of the p53 transcription factor includes modulation of cellular level of p53, as well as other biological or cellular activities of the p53 transcription factor. The term “p53 bioactivity” or “biological activity of p53” refers to the biochemical and physiological roles played by the p53 transcription factor in regulating cellular processes. The p53 transcription factor is involved in a very broad range of biological pathways and cellular activities (see, e.g., Almog et al., Biochim Biophys Acta, 1378:R43-R54, 1998). The broad spectrum of p53 bioactivities has been disclosed in the literature and in the present invention (e.g., Section II above and references cited therein). For example, p53 plays important roles in regulating cell growth arrest or apoptosis. Induction of p53 by DNA damage is linked to DNA repair and cellular development or differentiation. In addition, p53 participates in cytoskeletal modifications. Activation of the p53 pathway also leads to trans-regulation of expression of numerous target genes (p53 responsive genes), changes in cell adhesion, and secretion of extracellular factors (e.g., growth inhibitors).

Thus, p53 bioactivities to be monitored in this screening step include, but are not limited to, transcription or translation of p53, cellular level of p53, enzymatic or non-enzymatic modification (e.g., phosphorylation) of p53, tetramer formation, binding characteristics (e.g., binding to a target transcription regulatory element), regulation of expression of p53 responsive genes, interaction with another regulatory protein or molecule that is required for a p53 bioactivity (e.g., suppressor c-Abl or replication protein A), regulation of cellular proliferation or cell adhesion, or regulation of cell growth or apoptosis. All these bioactivities can be tested in the presence of a modulating agent that has been identified to bind to and/or modulate a p53-modulatory polypeptide.

2. Screening for p53 modulators

Modulation of cellular level or other bioactivities of the p53 transcription factor can be determined in a non-cell based assay system or cell-based assays, similar to the first screening step for identifying modulators of p53-modulatory polypeptides. Using eukaryotic in vitro transcription systems, effects of test agents on p53 level or activities can be tested by directly measuring in the presence of the test agents expression or cellular level of p53, or its transcription regulating activity. Because the test agent is likely to exert its modulatory effect on p53 by modulating a p53-modulatory polypeptide, the p53-modulatory polypeptide is typically also present in the assay system. In some embodiments, modulation of p53 cellular level can be examined using methods similar to that described in Example 2 below.

With cell-based assays, vectors expressing a reporter gene or other linked polynucleotides under the control of a transcription regulatory element of the p53 gene (for assaying modulation of p53 expression activity) or a p53 recognition sequence (for assaying modulation of p53 transcription regulating activities) are introduced into appropriate host cells. Modulation of p53 activities are typically examined by measuring expression of the reporter genes or other linked polynucleotides. An altered activity of the reporter gene (e.g., its expression level) in the presence of a test agent would indicate that the test agent is a modulator of p53.

If a p53 recognition sequence is used in the expression vector, an observed modulation of the reporter gene could be due to a direct interaction between the test agent with the expression vector. The modulation could also be due to an altered activity of endogenous p53 (e.g., DNA-binding activity or p53 cellular level) as a result of the presence of the test agent. The test agent's activity on the endogenous p53 could be direct, e.g., by interacting directly with p53, or indirect, e.g., through interacting with another molecule (e.g., a p53 modulatory polypeptide) that in turn binds to the p53 transcription regulatory element (e.g., a p53 recognition sequence). If the test agent was first identified to modulate a p53-modulatory polypeptide in the first screening step, its modulation on p53 activities or cellular level is likely to be indirect (i.e., through its interaction with the p53-modulatory polypeptide).

Various assays for analyzing p53 bioactivities have been described in the art and can be readily employed to screen for test agents that modulate p53 activities. For example, expression of p53 or cellular levels of p53 can be measured using routinely practiced methods (e.g., Sambrook et al., supra; and Ausubel et al., supra), as well as numerous methods described in the literatures (e.g., Sanchez-Prieto et al., Oncogene 11: 675-82, 1995; Lu et al., Oncogene 13: 413-8, 1996; and Furuta et al., Biochem J. 365: 639-48, 2002). Modulation of various biological activities of p53 by a test agent can also be assayed in accordance with many methods that have been disclosed in the art, e.g., Roemer et al., Proc Natl Acad Sci USA 90: 9252-6, 1993; Crook et al., Oncogene 9: 1225-30, 1994; Sabbatini et al., Mol Cell Biol 15: 1060-70, 1995; Canman et al., Genes Dev 9: 600-11, 1995; Gobert et al., Biochemistry 35: 5778-86, 1996; Lill et al., Nature 387: 823-7, 1997; Sabapathy et al., EMBO J. 16: 6217-29, 1997; Elmore et al., Proc Natl Acad Sci USA, 94: 14707-12, 1997; Hosokawa et al., Endocrinology 139: 4688-700, 1998; Fan et al., Mol Pharmacol 56: 966-72, 1999; Yang et al., J Leukoc Biol. 68: 916-22, 2000; and Appella, Eur J Biochem 268: 2763, 2001.

For example, similar to the first screening step, modulation of expression of a p53 responsive gene can be examined in a cell-based system by transient or stable transfection of an expression vector into cultured cell lines. Assay vectors bearing a p53 recognition sequence operably linked to reporter genes can be transfected into any mammalian cell line (e.g., HCT116 cell line as described in the Examples) for assays of promoter activity. General methods of cell culture, transfection, and reporter gene assay have been described in the art, e.g., Ausubel, supra; and Transfection Guide, Promega Corporation, Madison, Wis. (1998). Any readily transfectable mammalian cell line may be used to assay p53 promoter, e.g., HCT116, HEK 293, MCF-7, and HepG2 are all suitable cell lines.

Constructs containing a p53 recognition sequence (or a transcription regulatory element of the p53 gene) operably linked to a reporter gene can be prepared using only routinely practiced techniques and methods of molecular biology (see, e.g., Sambrook et al. and Ausubel et al., supra). Alternatively, expression vectors containing a reporter gene under the control of p53 response elements can also be obtained commercially (e.g., from Stratagene, San Diego, Calif.; see the Example below). P53 binding sites have been identified in a great number of p53 responsive genes. For example, p53 recognition sequences in p53 responsive genes have been disclosed in Yuan et al., Biochem Biophys Res Commun. 191: 662-8, 1993; Juven et al., Oncogene 8: 3411-6, 1993; Osifchin et al., J Biol Chem. 269: 6383-9, 1994; Jackson et al., Gene Expr. 5: 19-33, 1995; Strauss et al., Biochem Biophys Res Commun 217: 825-31, 1995; Jackson et al., FEBS Lett 406: 271-4, 1997; Mathupala et al., J Biol Chem 272: 22776-80, 1997; Schafer et al., FEBS Lett 436:139-43, 1998; and Szak et al., Mol Cell Biol. 21: 3375-86, 2001). Any of these transcription regulatory sequences can be employed in the present invention to study a test agent's ability to modulate p53 transcription regulating function. When the test agent is assayed for ability to modulate expression level of the p53 gene, transcription regulatory elements of the p53 gene can be used in the screening assay. Transcription regulatory elements of the p53 gene have also been well known and characterized in the art, e.g., as disclosed in Reisman et al., Proc Natl Acad Sci USA 85: 5146-50, 1988; Tuck et al., Mol Cell Biol. 9: 2163-72, 1989; Reisman et al., Cell Growth Differ. 4: 57-65, 1993; and Roy et al., Oncogene 13: 2359-66, 1996.

When inserted into the appropriate host cell, the transcription regulatory elements in the expression vector induces transcription of the reporter gene by host RNA polymerases. Reporter genes typically encode polypeptides with an easily assayed enzymatic activity that is naturally absent from the host cell. Typical reporter polypeptides for eukaryotic promoters include, e.g., chloramphenicol acetyltransferase (CAT), firefly or Renilla luciferase, beta-galactosidase, beta-glucuronidase, alkaline phosphatase, and green fluorescent protein (GFP).

Transcription driven by p53 response elements may also be detected by directly measuring the amount of RNA transcribed from the reporter gene. In these embodiments, the reporter gene may be any transcribable nucleic acid of known sequence that is not otherwise expressed by the host cell. RNA expressed from constructs containing a p53 response element may be analyzed by techniques known in the art, e.g., reverse transcription and amplification of mRNA, isolation of total RNA or poly A⁺ RNA, northern blotting, dot blotting, in situ hybridization, RNase protection, primer extension, high density polynucleotide array technology and the like. These techniques are all well known and routinely practiced in the art.

In addition to reporter genes, vectors for assaying expression under the control of a p53 recognition sequence can also comprise elements necessary for propagation or maintenance in the host cell, and elements such as polyadenylation sequences and transcriptional terminators to increase expression of reporter genes or prevent cryptic transcriptional initiation elsewhere in the vector. Exemplary assay vectors are the pGL3 series of vectors (Promega, Madison, Wis.; U.S. Pat. No. 5,670,356), which include a polylinker sequence 5′ of a luciferase gene. P53 response elements may be inserted into the polylinker sequence and tested for luciferase activity in the appropriate host cell. Assay vectors may also comprise additional enhancer or promoter sequences, depending on whether the transcription regulatory elements are sufficient to drive transcription of the reporter genes. For example, in addition to the p53 recognition sequence, the expression vectors can contain additional promoter sequence such as a minimal promoter (e.g., a promoter derived from Herpes simplex virus thymidine kinase) as discussed in Example 1.

If the p53 transcription regulatory sequence in the vector does not contain transcription initiation elements, an assay vector such as pGL3-Promoter may be used. This vector has transcription initiation elements from the SV40 promoter. In such vectors, transcription initiates from a heterologous site but the rate of transcription is increased by the presence of linked p53 response elements.

3. Agents Modulating p53 Responsive Genes

Other than monitoring p53 transcription regulating activity, a test agent that modulates a p53-modulatory polypeptide can be further screened for ability to modulate cellular proliferation through modulating p53 activity. In some methods, the test agent can be identified based on modulation of a cellular proliferation phenotype, e.g., inhibition of cell proliferation, cell or tumor growth arrest, or cell death. As discussed above, p53 activities in modulating cellular proliferation are well known, and methods for measuring such activities have also been described in the art (Sawhney et al., J Hum Hypertens 11: 611-4, 1997; Moretti et al., Oncogene 14: 729-40, 1997; and Onodera et al., Am J Dermatopatho 18: 580-8, 1996).

In some methods, a test agent modulating a p53-modulatory polypeptide and/or the p53 transcription factor can be further examined for effects on expression of p53 responsive genes. Expression of a great number of genes is known to be regulated by the p53 transcription factor. These genes are involved in many important cell processes such as DNA synthesis and repair processes, RNA transcription, and cisplatin resistance. For example, p53 responsive genes include: c-fos gene (Kley et al., Nucleic Acids Res, 20: 4083-7, 1992); herpes simplex thymidine kinase gene (Yuan et al., Biochem Biophys Res Commun 191: 662-8, 1993); placental transforming growth factor-beta (Wong et al., J Biol Chem: 277: 26699-707, 2002); Ku86 autoantigen related protein-1 gene (Braastad et al., Nucleic Acids Res 30: 1713-24, 2002); human bax genes (Thomborrow et al., Oncogene 21: 990-9, 2002); the hMSH2 gene (Warnick et al., J Biol Chem 276: 27363-70, 2001); glucocorticoid-inducible protein kinase (sgk) gene (Maiyar et al., Mol Endocrinol 11: 312-29, 1997); the bcl-2 gene (Miyashita et al., Cancer Res 54: 3131-5, 1994). Additional genes regulated by p53 include p21 (Waf1), Clp1, MDM2, GADD45, Cyclin G, IGF-BP3, and those described in U.S. Pat. No. 6,020,135.

### IV. Modulation of p53 Activity In Vivo

The present invention provides compositions and methods for modulating activities of the p53 transcription factor in a cell, and for modulating cellular proliferation. As a consequence of the connection between cellular proliferation and the p53 transcription factor, modulation of cellular levels or other bioactivities of the p53 transcription factor can lead to modulation of cellular proliferation. Modulation by the p53 modulators of the present invention (polypeptides or other molecules) can act through a number of mechanisms. The modulation can either be a decrease or an increase in the p53 promoter activity. For example, expression of p53 may be decreased or increased by binding of a p53 modulator to its promoter sequence. In some methods, modulation of cellular proliferation by p53 modulators of the present invention is achieved through modulating other biological activities of p53, e.g., its transcription regulating activity.

To modulate p53 activity in vivo, a cell can be contacted with any a number of the p53 modulators identified in accordance with the present invention. In some methods, a modulator of p53 of the present invention is introduced directly to a subject (e.g., a human or a non-human animal). In some methods, a polynucleotide encoding a modulator of p53 of the present invention is introduced by retroviral or other means (as detailed below). For example, the polynucleotides shown in Tables 1 and 2 or their fragments can be used to modulate p53 activity in vivo.

Activities of p53 modulators of the present invention can be examined or further verified in vivo by employing transgenic animals. Accordingly, transgenic animals with integrated p53 response elements can be used to assay modulation of p53 activities in vivo. Transgenic animals (e.g., transgenic mice) harboring p53 recognition sequences can be generated according to methods well known in the art. For example, techniques routinely used to create and screen for transgenic animals have been described in, e.g., see Bijvoet (1998) Hum. Mol. Genet. 7:53-62; Moreadith (1997) J. Mol. Med. 75:208-216; Tojo (1995) Cytotechnology 19:161-165; Mudgett (1995) Methods Mol. Biol. 48:167-184; Longo (1997) Transgenic Res. 6:321-328; U.S. Pat. No. 5,616,491 (Mak, et al.); U.S. Pat. Nos. 5,464,764; 5,631,153; 5,487,992; 5,627,059; 5,272,071; and, WO 91/09955, WO 93/09222, WO 96/29411, WO 95/31560, and WO 91/12650.

In some embodiments, p53 recognition sequences operably linked to a reporter gene are injected into the embryo of a developing animal (typically a mouse) to generate a transgenic animal. Once integration of the transgene has been verified, tissues of the animal (e.g., lymphoid tissues) are then assayed for expression of the transgene. For example, where the p53 recognition sequence is linked to a reporter gene, tissues of the transgenic animal may be assayed either for reporter gene RNA or for the enzymatic activity of the reporter polypeptide.

In the transgenic animals, p53 recognition sequences will generally display appropriate regulation regardless of the site of transgene integration. However, constructs comprising the regulatory sequences can also be flanked by insulator elements to ensure complete independence from position effects (see Bell et al., Science 291:447-50, 2001).

### V. Therapeutic Applications

The p53 transcription factor plays an important role in cellular proliferation and tumor suppression. Many clinical conditions or disease states are linked to abnormal cell proliferation. Such disease states and disorders include those involving the hyperproliferation of cells such as, e.g., a tumor (neoplasm) or malignant tumor. Tumors are abnormal growths resulting from the hyperproliferation of cells. Cells that proliferate to excess but stay put form benign tumors, which can typically be removed by local surgery. In contrast, malignant tumors or cancers comprise cells that are capable of undergoing metastasis, i.e., a process by which hyperproliferative cells spread to, and secure themselves within, other parts of the body via the circulatory or lymphatic system (see, generally, Chapter 16 In: Molecular Biology of the Cell, Alberts et al., eds., pp. 891-950, Garland Publishing, Inc., New York, 1983).

Accordingly, the invention provides therapeutic compositions and methods for preventing or treating diseases and conditions due to abnormal cellular level or other biological activities of p53. The compositions and methods are useful for treating or modulating various hyperproliferative disorders or diseases, such as various cancers. Modulation of p53 activity or cellular levels is also useful for preventing or modulating the development of such diseases or disorders in an animal suspected of being, or known to be, prone to such diseases or disorders. The pharmaceutical compositions can comprise a polypeptide modulator of p53 identified in accordance with the present invention (e.g., as shown in Tables 1 and 2), an antibody against such modulators, or other modulators disclosed herein which directly or indirectly modulate p53 activities.

A. Examples of Disease and Conditions Amenable to Treatment

A great number of diseases and conditions are amenable to treatment with methods and compositions of the present invention. Examples of tumors that can be treated with methods and compositions of the present invention include but are not limited to skin, breast, brain, cervical carcinomas, testicular carcinomas. They encompass both solid tumors and metastatic tumors. Cancers that can be treated by the compositions and methods of the invention include cardiac cancer (e.g., sarcoma, myxoma, rhabdomyoma, fibroma, lipoma and teratoma); lung cancer (e.g., bronchogenic carcinoma, alveolar carcinoma, bronchial adenoma, sarcoma, lymphoma, chondromatous hamartoma, mesothelioma); various gastrointestinal cancer (e.g., cancers of esophagus, stomach, pancreas, small bowel, and large bowel); genitourinary tract cancer (e.g., kidney, bladder and urethra, prostate, testis; liver cancer (e.g., hepatoma, cholangiocarcinoma, hepatoblastoma, angiosarcoma, hepatocellular adenoma, hemangioma); bone cancer (e.g., osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma and giant cell tumors); cancers of the nervous system (e.g., of the skull, meninges, brain, and spinal cord); gynecological cancers (e.g., uterus, cervix, ovaries, vulva, vagina); hematologic cancer (e.g., cancers relating to blood, Hodgkin's disease, non-Hodgkin's lymphoma); skin cancer (e.g., malignant melanoma, basal cell carcinoma, squamous cell carcinoma, Karposi's sarcoma, moles dysplastic nevi, lipoma, angioma, dermatofibroma, keloids, psoriasis); and cancers of the adrenal glands (e.g., neuroblastoma).

Disease states other than cancer which can be treated by the methods and compositions also include restenosis, autoimmune disease, arthritis, graft rejection, inflammatory bowel disease, proliferation induced after medical procedures such as surgery, angioplasty, and the like. Other diseases and conditions are also know in the art which has implicated abnormal p53 activities. In some methods, cells not in a hyper or hypo proliferation state (abnormal state) are the subject of treatment. For example, during wound healing, the cells may be proliferating “normally”, but proliferation enhancement may be desired. Similarly, in the agriculture arena, cells may be in a “normal” state, but proliferation modulation may be desired to enhance a crop by directly enhancing growth of a crop, or by inhibiting the growth of a plant or organism which adversely affects the crop. Thus, therapeutic applications of the present invention include treatment of individuals or agricultural crops with any one of these disorders or states.

B. Administration of p53 Modulators

1. Pharmaceutical Compositions

The p53 modulators of the present invention can be directly administered under sterile conditions to the subject to be treated. The modulators can be administered alone or as the active ingredient of a pharmaceutical composition. Therapeutic composition of the present invention can be combined with or used in association with other therapeutic agents. For example, a subject may be treated with conventional chemotherapeutic agents, particularly those used for tumor and cancer treatment. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine (CA), 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, vincristine, vinblastine, etoposide, trimetrexate, teniposide, cisplatin and diethylstilbestrol (DES). See, generally, The Merck Manual of Diagnosis and Therapy, 15th Ed., pp. 1206-1228, Berkow et al., eds., Rahay, N.J., 1987). When used with the compounds of the invention, such chemotherapeutic agents may be used individually, sequentially, or in combination with one or more other such chemotherapeutic agents.

Pharmaceutical compositions of the present invention typically comprise at least one active ingredient together with one or more acceptable carriers thereof. Pharmaceutically carriers enhance or stabilize the composition, or to facilitate preparation of the composition. Pharmaceutically acceptable carriers are determined in part by the particular composition being administered (e.g., nucleic acid, protein, modulatory compounds or transduced cell), as well as by the particular method used to administer the composition. They should also be both pharmaceutically and physiologically acceptable in the sense of being compatible with the other ingredients and not injurious to the subject. This carrier may take a wide variety of forms depending on the form of preparation desired for administration, e.g., oral, sublingual, rectal, nasal, or parenteral. For example, the p53 modulator can be complexed with carrier proteins such as ovalbumin or serum albumin prior to their administration in order to enhance stability or pharmacological properties.

There are a wide variety of suitable formulations of pharmaceutical compositions of the present invention (see, e.g., Remington: The Science and Practice of Pharmacy, Mack Publishing Co., 20^{th} ed., 2000). Without limitation, they include syrup, water, isotonic saline solution, 5% dextrose in water or buffered sodium or ammonium acetate solution, oils, glycerin, alcohols, flavoring agents, preservatives, coloring agents starches, sugars, diluents, granulating agents, lubricants, and binders, among others. The carrier may also include a sustained release material such as glyceryl monostearate or glyceryl distearate, alone or with a wax.

The pharmaceutical compositions can be prepared in various forms, such as granules, tablets, pills, suppositories, capsules, suspensions, salves, lotions and the like. The concentration of therapeutically active compound in the formulation may vary from about 0.1-100% by weight. Therapeutic formulations are prepared by any methods well known in the art of pharmacy. See, e.g., Gilman et al., eds., Goodman and Gilman's: The Pharmacological Bases of Therapeutics, 8th ed., Pergamon Press, 1990; Remington: The Science and Practice of Pharmacy, Mack Publishing Co., 20^{th} ed., 2000; Avis et al., eds., Pharmaceutical Dosage Forms: Parenteral Medications, published by Marcel Dekker, Inc., N.Y., 1993; Lieberman et al., eds., Pharmaceutical Dosage Forms: Tablets, published by Marcel Dekker, Inc., N.Y., 1990; and Lieberman et al., eds., Pharmaceutical Dosage Forms: Disperse Systems, published by Marcel Dekker, Inc., N.Y., 1990.

2. Modes of Administration

The therapeutic formulations can be delivered by any effective means which could be used for treatment. Depending on the specific p53 modulators to be administered, the suitable means include oral, rectal, vaginal, nasal, pulmonary administration, or parenteral (including subcutaneous, intramuscular, intravenous and intradermal) infusion into the bloodstream.

For parenteral administration, p53 modulators (including polynucleotides encoding p53 modulators) of the present invention may be formulated in a variety of ways. Aqueous solutions of the modulators may be encapsulated in polymeric beads, liposomes, nanoparticles or other injectable depot formulations known to those of skill in the art. The nucleic acids may also be encapsulated in a viral coat.

Additionally, the compounds of the present invention may also be administered encapsulated in liposomes. The compositions, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such a diacetylphosphate, stearylamine, or phosphatidic acid, and/or other materials of a hydrophobic nature.

The compositions may be supplemented by active pharmaceutical ingredients, where desired. Optional antibacterial, antiseptic, and antioxidant agents may also be present in the compositions where they will perform their ordinary functions.

3. Dosages

The therapeutic formulations can conveniently be presented in unit dosage form and administered in a suitable therapeutic dose. A suitable therapeutic dose can be determined by any of the well known methods such as clinical studies on mammalian species to determine maximum tolerable dose and on normal human subjects to determine safe dosage. Except under certain circumstances when higher dosages may be required, the preferred dosage of a p53 modulator usually lies within the range of from about 0.001 to about 1000 mg, more usually from about 0.01 to about 500 mg per day.

The preferred dosage and mode of administration of a p53 modulator can vary for different subjects, depending upon factors that can be individually reviewed by the treating physician, such as the condition or conditions to be treated, the choice of composition to be administered, including the particular p53 modulator, the age, weight, and response of the individual subject, the severity of the subject's symptoms, and the chosen route of administration. As a general rule, the quantity of a p53 modulator administered is the smallest dosage which effectively and reliably prevents or minimizes the conditions of the subjects. Therefore, the above dosage ranges are intended to provide general guidance and support for the teachings herein, but are not intended to limit the scope of the invention.

In some applications, a first p53 modulator is used in combination with a second p53 modulator in order to modulate p53 molecules to a more extensive degree than cannot be achieved when one p53 modulator is used individually.

ps C. Delivery of Polynucleotides Encoding p53 Modulators

In some methods of the present invention, polynucleotides encoding p53 modulators of the present invention (e.g., those listed in Tables 1 and 2 or fragments thereof, and substantially identical polynucleotides) are transfected into cells for therapeutic purposes in vitro and in vivo. These polynucleotides can be inserted into any of a number of well-known vectors for the transfection of target cells and organisms as described below. The nucleic acids are transfected into cells, ex vivo or in vivo, through the interaction of the vector and the target cell. The compositions are administered to a subject in an amount sufficient to elicit a therapeutic response in the subject.

Such gene therapy procedures have been used to correct acquired and inherited genetic defects, cancer, and viral infection in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11:162-166 (1993); Mulligan, Science 926-932 (1993); Dillon, TIBTECH 11:167-175 (1993); Miller, Nature 357:455-460 (1992); Van Brunt, Biotechnology 6(10):1149-1154 (1998); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology (Doerfler & Böhm eds., 1995); and Yu et al., Gene Therapy 1:13-26 (1994)).

Delivery of the gene or genetic material into the cell is the first step in gene therapy treatment of disease. A large number of delivery methods are well known to those of skill in the art. Preferably, the polynucleotides are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell.

Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. No. 5,049,386, U.S. Pat. No. 4,946,787; and U.S. Pat. No. 4,897,355 and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to subjects (in vivo) or they can be used to treat cells in vitro and the modified cells are administered to subjects (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vector that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In particular, a number of viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent.

pLASN and MFG-S are examples are retroviral vectors that have been used in clinical trials (Dunbar et al., Blood 85:3048-305 (1995); Kohn et al., Nat. Med. 1:1017-102 (1995); Malech et al., Proc. Natl. Acad. Sci. U.S.A. 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors (Ellem et al., Immunol Immunother. 44(1):10-20 (1997); Dranoff et al., Hum. Gene Ther. 1:111-2 (1997)).

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., Proc. Natl. Acad. Sci. U.S.A. 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual subject, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual subject (e.g., lymphocytes, bone marrow aspirates, tissue biopsy) or universal donor hematopoietic stem cells, followed by reimplantation of the cells into a subject, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., subject). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from subjects).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

### EXAMPLES

The following examples are offered to illustrate, but not to limit the present invention.

### Example 1

Modulation of Expression from a p53 Enhancer Element

This Example describes identification of various p53-modulatory polypeptides that regulate expression of a reporter gene under the control of a p53 recognition sequence. P53 recognition sequences control transcription of a great number of genes regulated by p53 (p53 responsive genes).

An arrayed and annotated cDNA library in a mammalian expression vector was interrogated for modulators of p53 activity. The library, consisting of approximately 20,000 full-length human cDNAs was spotted in 384 well plates such that each well contained an individual cDNA with known identity. In a semi-automated process, cDNAs were incubated with a non-liposomal transfection reagent (Fugene, Roche Applied Science, Indianapolis, Ind.) and a p53-luciferase reporter vector (Stratagene, San Diego, Calif.). This vector contains fourteen tandem copies of a 17-base pair p53 response element having the sequence and a basic promoter element (TATA box).

Then human colon cancer derived cells (HCT116, p53+/+) were introduced into each well to complete the transfection procedure. After 2 days of incubation at 37 C., 5% CO2, equal volumes of Bright-glo reagent (Promega, Madison, Wis.) was added to each well and relative luminescence was quantitated using an Acquest (LJL Biosystems, Sunnyvale, Calif.) plate reader.

After executed the assay in duplicate, plate data were normalized to a mean value and compared across the library (20,000 wells). Approximately 90 cDNAs with mean activity values > or < 4 standard deviations from the whole experimental mean were selected from the library, and were amplified and isolated utilizing commercially available DNA isolation reagents (Qiagen, Germany). These samples were reconfirmed utilizing the methods outlined above. Tables 1 and 2 above provide cDNAs that possess confirmed modulatory (down-regulating or up-regulating) activity on the reporter gene expression under control of the p53 recognition sequence. Several known p53 modulators, e.g., HIF1a and ARF, were also uncovered in the screen, validating the authenticity of the screen.

### Example 2

Confirmation of p53-Regulatory Activities of the Novel p53 Modulators

This Example describes confirmation of p53-modulatory function of some of the novel p53 modulators discussed above. These include eight p53-inducing genes, HEY1, OSR1, HES1, AP-4, NR2F2, SFRS10, SMT3 and hypothetic gene FLJ11339, which were detected to have the most potent induction. Also analyzed are two p53-inhibitory genes, M17S2 and cathepsin B, which have most potent suppression of the reporter plasmid. Among the positive regulators, four genes, Hey1, Hes1, Osr1 and AP-4 belong to bHLH (basic helix-loop-helix) superfamily. The bHLH proteins are a family of transcription factors that regulate various biological processes. The two negative regulators, M17S2 and cathepsin B, were both implicated in tumor development. M17S2 contains a B-box/coled coil motif, which is commom in genes with transformation potentials. M17S2 is also a surface marker in ovarian cancer. The expression of M17S2 was found to be inversely correlated to the survival length in renal cell carcinoma patients, predicting a significantly higher probability of death. Cathepsin B is a lysosomal cysteine protease. Overexpression of the protein has been associated with ovarian cancer, adenocarcinoma and other tumors.

In order to confirm the induction of p53 reporter plasmid is indeed p53-dependent, there genes and the reporter plasmid along with β-gal plasmid were transfected into isogenic HCT116 p53+/+ and p53−/− cells. This pair of cell lines has identical genetic background except p53 status. When positive regulators are expressed, luminescence signals were 3-8 fold higher than control in wild type HCT116 cells, but showed no difference in p53−/− cells, indicating the activation of reporter plasmid is p53-dependent. Two negative regulators decrease signals about 60-70% whereas control hDM2 inhibit about 75%. We also tested a control reporter plasmid that contains minimum TATA box but no p53 binding element. The co-transfection of this control plasmid with these hits did not give higher signals than vector, further demonstrating these genes are not general transcription activators.

The induction of p53 is also confirmed by immunoblot assay. When transfected with HEY1, OSR1, HES1, AP-4 and NR2F2, the p53 protein level is significantly higher in HCT116 cells and its downstream target P21 protein level is also elevated. These results demonstrate that cDNAs discovered through the screen are bona fide positive regulators of endogenous p53 in mammalian cells.

To differentiate the activation occurs at the transcriptional level or post-transcriptional level, p53 promoter reporter plasmid was co-transfected with these molecules into HCT116 p53−/− cells and luminescence signals were measured after 48 hrs. NR2F2 and SRSF 10 showed a dramatic increase of signals, suggesting these two genes increase p53 activity by positive regulation of p53 transcription whereas the other six hits (i.e., HEY1, OSR1, AP-4, HES1, SMT3, FLJ11339) function at the post-transcriptional level of p53.

### Example 3

Characterization of Novel p53 Modulators

This Example describes further characterization of a few members of the novel p53 modulators discussed above. Given the role of p53 in tumor suppression, we tested the ability of these molecules to inhibit the transformation of Ras and Myc in mouse embryonic fibroblasts (MEFs). It has been shown that inhibition of p53 is required for Ras transformation in MEFs and reactivation of p53 can inhibit the transformation. The retroviruses containing Ras, Myc and the genes were co-infected in MEFs and p53−/− MEFs and the foci formation were measured by crystal violet staining. The virus containing HEY1 was able to inhibit foci formation by Ras and Myc in wild type MEFs, but not in p53−/− MEFs. These results demonstrated that the transformation suppression by HEY 1 was mediated through p53 activation.

Since hDM2, human homologue of MDM2, is a major regulator of p53 at the post-transcriptional level, we tested the effects of these genes on hDM2. hDM2 promoter reporter construct was co-transfected with these molecules into HCT 116 and HEK293 cells. Four basic helix-loop-helix genes, Hey1, OSR1, HES1 and AP-4, inhibited luminescence activity of hDM2 reporter 3-7 fold in both cells lines, whereas DCP 1, an unrelated bHLH gene, showed no inhibitory activity. Immunoblot assay from the cell extracts confirmed that hDM2 protein level is decreased following the transfection of these genes. Quantitative RT-PCR showed that these four bHLH genes inhibit hDM2 activity at the transcriptional level. We also tested other unrelated bHLH repressor DCP1 and activator Bmal and Clock. DCP1 did not inhibit hDM2, demonstrating the activation specificity of HEY1, HES1, OSR1 and AP-4. Interestingly, we found that co-transfection of bHLH circadian clock genes, Bmal1 and Clock, induce three-fold increase of luminescence signals on hDM21uc reporter in HCT116 cells, suggesting that hDM2 may also be regulated by bHLH activators. It has been shown that Hey1 and Hes1 are able to bind to specific sequence E box (CANNTG) and N box (CACNAG). We search the promoter region in the reporter plasmid and found one E box and one N box. Gel shift assay was performed and failed to show that HEY1 and HES1 protein bind to these specific sequence, suggesting these proteins may function by binding to the other members of bHLH family that serve as hDM2 activator.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, ATCC deposits, patents and patent applications cited herein are hereby expressly incorporated by reference in their entirety and for all purposes as if each is individually so denoted.

We claim:

1. A method for identifying an agent that modulates a p53 bioactivity, the method comprising:
(a) assaying a biological activity of a p53-modulatory polypeptide encoded by a polynucleotide selected from the members listed in Table 1 and Table 2, or a fragment of said polypeptide, in the presence of a test agent to identify one or more modulating agents that modulate the biological activity of the polypeptide; and
(b) testing one or more of the modulating agents for ability to modulate a p53 bioactivity.

2. The method of claim 1, wherein the p53-modulatory polypeptide up-regulates p53 and is selected from the members listed in Table 2.

3. The method of claim 2, wherein the p53-modulatory polypeptide is selected from group consisting of HEY1, OSR1, HES1, AP-4, NR2F2, SFRS10, SMT3 and FLJ11339.

4. The method of claim 1, wherein the p53-modulatory polypeptide down-regulates p53 and is selected from the members listed in Table 1.

5. The method of claim 4, wherein the p53-modulatory polypeptide is selected from group consisting of M17S2 and cathepsin B.

6. The method of claim 1, wherein (b) comprises testing the modulating agents for ability to modulate p53 in regulating expression of a p53 responsive gene.

7. The method of claim 1, wherein (b) comprises testing the modulating agents for ability to modulate cellular level of p53.

8. The method of claim 1, wherein the p53-modulatory polypeptide is a kinase and the biological activity is phosphorylation of a second polypeptide.

9. The method of claim 8, wherein the second polypeptide is p53 or a fragment of p53.

10. The method of claim 1, wherein the p53-modulatory polypeptide is a protease and the biological activity is proteolysis of a second polypeptide.

11. The method of claim 10, wherein the second polypeptide is p53 or a fragment of p53.

12. The method of claim 1, wherein the test agent modulates cellular level of the p53-modulatory polypeptide.

13. The method of claim 1, wherein the assaying of the biological activity of the p53-modulatory polypeptide occurs in a cell.

14. The method of claim 13, wherein the p53-modulatory polypeptide is expressed from said polynucleotide that has been introduced into the cell.

15. The method of claim 14, wherein the p53 bioactivity is inducing expression of a second polynucleotide that is operably linked to a p53 response element.

16. The method of claim 15, wherein the second polynucleotide encodes a reporter polypeptide.

17. The method of claim 16, wherein the testing for ability to modulate a p53 bioactivity comprises:
providing a cell or cell lysate that comprises the second polynucleotide that is operably linked to the p53 response element;
contacting the cell or cell lysate with the test agent; and
detecting an increase or decrease in expression of the second polynucleotide in the presence of the test agent compared to expression of the second polynucleotide in the absence of the test agent.

18. The method of claim 1, wherein the testing for ability to modulate a p53 bioactivity comprises contacting a cell or cell lysate with the test agent and determining cellular level of p53 or a fragment of p53.

19. The method of claim 1, wherein the testing for ability to modulate the p53 bioactivity comprises contacting a cell or cell lysate with the test agent and determining ability of p53 to bind to a second polynucleotide that comprises a p53 response element in the cell or cell lysate.

20. A method for identifying an agent that modulates cellular level of p53, the method comprising:
(a) assaying a biological activity of a p53-modulatory polypeptide encoded by a polynucleotide selected from the members listed in Tables 1 and 2, or a fragment of said polypeptide, in the presence of a test agent to identify a modulating agent that modulates the biological activity of the polypeptide; and
(b) testing the modulating agent for ability to modulate cellular level of p53.

21. The method of claim 20, wherein the p53-modulatory polypeptide is a transcription regulatory protein and the biological activity is transcription of a second polynucleotide.

22. The method of claim 21, wherein the p53-modulatory polypeptide is selected from the group consisting of HEY1, OSR1, HES1, AP-4, NR2F2, SFRS10, SMT3 and FLJ11339.

23. The method of claim 21, wherein the second polynucleotide encodes p53 or a fragment of p53.

24. The method of claim 20, wherein the testing comprises (i) contacting the modulating agent with a second polynucleotide operably linked to a transcription regulatory element of p53; and (ii) detecting a change in cellular level of a polypeptide encoded by said second polynucleotide relative to cellular level of the polypeptide encoded by the second polynucleotide in the absence of the modulating agent.

25. The method of claim 24, wherein the contacting occurs in a cell.

26. The method of claim 24, wherein said second polynucleotide encodes a reporter polypeptide.

27. A method of modulating a p53 bioactivity in a cell, comprising administering to the cell an effective amount of a p53 modulator identified in claim 1, thereby modulating the p53 bioactivity.

28. The method of claim 27, wherein said modulating is increasing cellular level of p53.
